(19) United States
(12) Patent Application Publication
(10) Pub. No.: US 2018/0317998 A1
(43) Pub. Date: Nov. 8, 2018

(54) RADIO FREQUENCY TREATMENT OF SWEAT GLANDS

(71) Applicant: Alma Lasers Ltd., Caesarea (IL)

(72) Inventors: Alexander Britva, Migdal Ha'Emek (IL); Ziv Karni, Kfar Shmaryahu (IL); Joseph Lepselter, Nordiya (IL)

(21) Appl. No.: 16/039,362

(22) Filed: Jul. 19, 2018

Related U.S. Application Data
(63) Continuation of application No. 14/488,274, filed on Sep. 16, 2014, now abandoned.
(60) Provisional application No. 61/878,109, filed on Sep. 16, 2013.

(51) Int. Cl. A61B 18/14

(52) U.S. Cl. CPC A61B 18/14; A61N 1/322; A61B 2018/1405

(57) ABSTRACT

Apparatuses and methods for thermally treating sweat glands in a volume of skin of a patient. A unipolar RF handpiece comprises a unipolar RF electrode and a bipolar RF handpiece comprises a supply electrode and a ground electrode, separated by an insulator. The unipolar RF handpiece applies RF waves into the volume of skin to dielectrically heat the sweat glands and the bipolar RF handpiece introduces RF current dielectrically heat the sweat glands.

In some embodiments, the electrodes of the bipolar RF handpiece are arranged coaxially and are adjustable to allow for adjusting the depth of the RF current. In some embodiments the RF waves are applied first, heating the sweat glands and thus increasing their water content so the RF current can be applied to complete thermal disruption or destruction of the sweat glands without disrupting surrounding tissue. In some embodiments, after the RF waves are applied, the RF current is applied to an upper part of the volume, thereby slowing conductive or convective cooling of the remainder of the volume below it.

### CROSS REFERENCE TO RELATED APPLICATIONS

This patent application is a continuation of U.S. nonprovisional patent application Ser. No. 14/488,274 filed on Sep. 16 2014, which claims the benefit of U.S. provisional patent application No. 61/878,109 filed on Sep. 16 2013, both of which are hereby incorporated by reference in their entirety.

### BACKGROUND OF THE INVENTION

The invention relates to the application of radio frequency electromagnetic energy for treatment of sweat-related medical conditions by thermal disruption, or destruction, of sweat glands, including apocrine, eccrine and apoeccrine glands.

US patent application publication 20100049178 discloses methods and related apparatuses for reducing sweat production by positioning an energy delivery device proximate to a skin tissue of the patient and delivering energy to sweat glands to halt secretion of sweat.

US patent application publication 20110190745 discloses methods and related apparatuses for reducing sweat production by causing thermal injury by delivering electromagnetic radiation to a target region of skin.

Such earlier proposed solutions for treating conditions such as hyperhidrosis (HH) and osmidrosis (OM) do not adequately resolve the dichotomy of applying sufficient energy to damage or destroy the sweat glands in an intracutaneous target area while seeking to avoid pain and damage to intervening tissue layers and to neighboring structures in the target area.

To allay pain and some damage, topical contact cooling elements have been added, however these are but partially effective.

To lessen damage to neighboring tissue structures, attempts have been made to increase the water content in the sweat glands and then apply microwave energy to dielectricly heat the water, thereby selectively heat the sweat glands. However, the methods of increasing the water content are either inefficient (injection) or damaging (application of the microwave energy for this purpose as well as for treatment). Furthermore, application of microwave frequencies for dielectric heating of tissue requires particular care and precision to avoid burning.

Thus, there remains a considerable need for apparatuses and methods that can thermally disable or destroy sweat glands with less pain and damage to other tissue.

### SUMMARY OF THE INVENTION

The present inventors have discovered that the above-mentioned problems can be resolved by application of bipolar and unipolar modes of radio frequency (RF) electromagnatic energy to thermally treat sweat glands.

In some embodiments, an apparatus is provided for thermally treating sweat glands in a volume of skin of a patient. The apparatus comprises a unipolar RF handpiece comprising a unipolar RF electrode and a bipolar RF handpiece comprising a supply electrode and a ground electrode, separated by an insulator. The unipolar RF handpiece is operable to introduce RF waves from an RF energy source through the unipolar electrode into the volume of skin to dielectrically heat the sweat glands, and the bipolar RF handpiece is operable to introduce RF current from the RF energy source through the supply electrode into the biological tissue and on to the ground electrode to dielectrically heat the sweat glands.

In some embodiments the supply electrode of the bipolar RF handpiece is arranged coaxially inside the ground electrode, forming a uniform, homogeneous distribution of the RF current introduced by the bipolar handpiece.

In some embodiments the distance between the supply electrode and the ground electrode of the bipolar RF handpiece is adjustable.

In some embodiments a method is provided for thermally treating sweat glands in a volume of skin of a patient, the method comprising applying RF waves to a first portion of the volume of skin to dielectrically heat the sweat glands, thereby increasing their water content; and applying RF current to a second portion of the volume of skin, thereby further dielectrically heating those heated sweat glands common to both the first and second portions of the volume.

In some embodiments the sweat glands heated by the RF current are disrupted or destroyed.

In some embodiments the disruption or destruction of the sweat glands by the RF current occurs before the surrounding and or intervening tissue reaches a disruptive temperature, with the result that the treatment is effectively selective for the sweat glands.

In some embodiments a method is provided for thermally treating sweat glands in a volume of skin of a patient, the method comprising: applying RF waves to at least a lower part of the volume of skin to dielectrically heat the sweat glands, thereby increasing their water content; and applying RF current to an upper part of the volume of skin, thereby slowing conductive or convective cooling of the heated lower part of the volume of skin through the upper part, so as to maintain the therapeutic heat longer in the volume without directly heating the entire volume.

Various objects, features, aspects, and advantages of the present invention will become more apparent from the following detailed description of preferred embodiments of the invention, along with the accompanying drawings in which like numerals represent like components.

### BRIEF DESCRIPTION OF THE DRAWINGS

The invention will now be described further, by way of example, with reference to the accompanying drawings, in which:

FIG. 1 is a perspective drawing of a handpiece for bipolar application of radio frequency electromagnetic energy in accordance with the present invention.

FIG. 2 is a perspective drawing of a handpiece for unipolar application of radio frequency electromagnetic energy in accordance with the present invention.

### DETAILED DESCRIPTION

The present invention relates to apparatuses and methods for thermally disrupting or destroying sweat glands via application of RF electromagnetic energy.

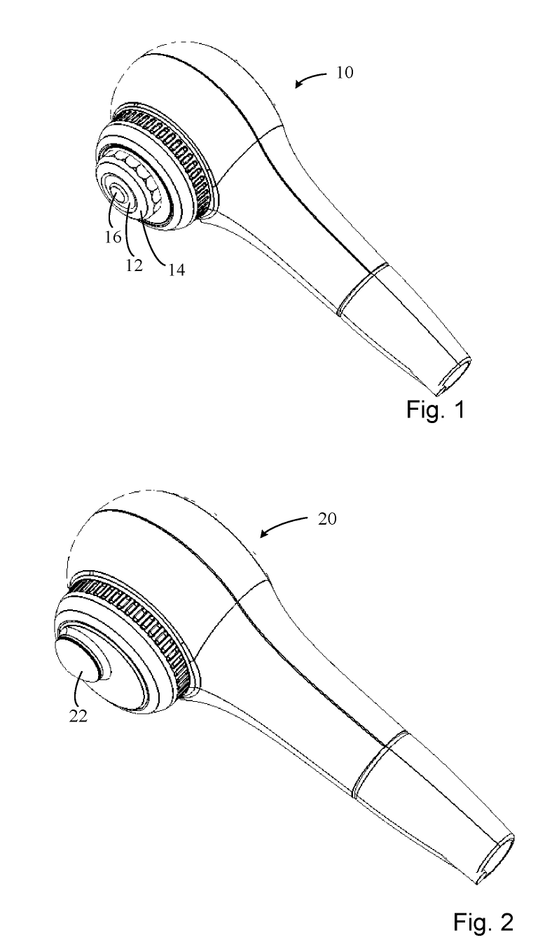

FIG. 1 illustrates a handpiece for bipolar application of RF energy 10, comprising two coaxial (concentric) electrodes: a central supply electrode 16 and a surrounding ground electrode 14, separated by an insulator 12.

Bipolar application of RF electromagnetic energy to heat human tissue is generally known in the art.

While other arrangements of the bipolar electrodes 14, 16 can be used, the particular coaxial arrangement of the embodiment in FIG. 1 ensures a uniform, homogeneous distribution of the bipolar current in contrast with distortion that can be caused by the electrode orientations found in other bipolar arrangements.

In some embodiments the penetration depth of the bipolar current can be modified by changing the distance between the supply electrode 16 and ground electrode 14, for example, by making the electrode diameters adjustable or by making the electrodes user swappable.

In some embodiments bipolar RF handpiece 10 is optimized for heating at a distance of 1 mm to 8 mm below the skin surface.

FIG. 2 illustrates a handpiece for unipolar application of RF energy 20, comprising a single unipolar electrode 22 for propagation of the waves of an output RF power signal.

Unipolar application of RF electromagnetic energy to heat human tissue is described in U.S. Pat. No. 7,630,774 and U.S. Pat. No. 8,150,532 granted to two of the present inventors, both of which patents are hereby incorporated by reference.

U.S. Pat. No. 7,630,774 and U.S. Pat. No. 8,150,532 disclose a system for heating biological tissue which uses the unipolar RF handpiece 20 to apply RF energy to the tissue. The same system can use bipolar RF handpiece 10.

The system described in U.S. Pat. Nos. 7,630,774 and 8,150,532 includes the RF unipolar handpiece 20, forming a single electrode 22 of the system, the single electrode 22 comprising a dielectric barrier, contactable with a surface of a biological tissue to be heated. The RF unipolar handpiece 20 is capable of delivering a desired amount of energy to a predetermined energy dissipation zone beneath the surface of the biological tissue, the selected target being positioned within the predetermined energy dissipation zone. An RF energy source produces an output RF power signal directed to the RF unipolar handpiece 20, the RF being radiatively or capacitively coupled by the applicator into the biological tissue. A phase shifter is provided which is controllable to vary the phase of the output RF power signal so that energy from it is concentrated primarily in the predetermined energy dissipation zone, which lies at a phase dependent depth beneath the surface of the biological tissue. An impedance matching network serves to match an impedance characteristic of the RF energy source and the phase shifter to the impedance of the biological tissue belonging to the subject so that the RF power signal may pass through the surface of the biological tissue without undergoing reflection. An RF resonator, located in the RF unipolar handpiece 20, is operative to accumulate and release the desired amount of energy cyclically whereby a significant portion of the energy of the RF signal is concentrated in the predetermined energy dissipation zone.

In operation, the RF unipolar handpiece 20 serves to convey the output RF power signal from the RF energy source through the surface of the biological tissue to the predetermined energy dissipation zone after the output has been processed by the phase shifter, the impedance matching network and the resonator. Operation of the system thereby produces a reverse thermal gradient in the biological tissue in that the surface is maintained at a lower temperature than the predetermined energy dissipation zone without using a cooling device for cooling the surface.

Both bipolar RF handpiece 20 and unipolar RF handpiece 10 apply dielectric heating of the skin by rotation of water molecules in alternating electromagnetic fields, whereby dissipation of some specific energy is converted to an increase in the temperature of the tissue. Dielectric heating effectiveness increases in tissues with higher water concentration.

Sweat glands, which are responsible for thermo-compensation of heating, are not distinguished from the surrounding tissues before the sweating process begins. Accordingly, dielectric treatment will tend to be most effective and selective in cases of strong hyperhidrosis (sweat glands working continuously) but may be less so in cases of moderate or weak hyperhidrosis.

Therefore, to implement selective deactivation (damage or destruction) of sweat glands, it is preferable to initiate their sweating function, thereby leading to increased water concentration in the glands and consequently their efficient thermal deactivation by RF energy before that energy thermally damages the surrounding tissues (dermis and hypodermis).

To this end, a method of the present invention is to apply preliminary deep heating of the deep dermis/upper hypodermis target area with unipolar RF handpiece 20 to raise the temperature in the target area to between 40 degrees celsius and 45 degrees celsius, stimulating a reaction of the sweat glands and increasing their water content. Thereupon, bipolar RF handpiece 10 treatment is applied to selectively damage the glands.

In some embodiments, the invention is capable of applying energy with power in the range of 100 to 400 watts at a frequency in the range of 30 MHz to 100 MHz.

In some embodiments, the frequency of the RF EM energy is an ‘industrial, scientific and medical’ (ISM) radio band center frequency of 40.680 MHz, hence the crest of the wave (i.e., quarter wavelength or lambda/4) is 1.84 m, which is close to average body length. In such cases, the body acts as an antenna, to attract maximum power.

In some embodiments, the unipolar RF handpiece 10 is first applied to raise the temperature of a volume of skin comprising sweat glands to a temperature that starts therapeutic degradation of the glands. The bipolar RF handpiece 20 is then applied to heat the upper part of the volume, thereby slowing conductive or convective cooling of the volume through that upper part, so as to maintain the therapeutic heat longer in the volume without directly heating the entire volume.

In some embodiments, the unipolar RF handpiece 10 is first applied to raise the temperature of a volume of skin comprising sweat glands to a temperature that induces production of sweat in the glands. The bipolar handpiece is then applied and achieves more effective heating due to the increased water content of the glands caused by the produced sweat.

In some embodiments, the aforementioned increased water content increases the selectivity of the procedure in that the glands are sooner heated than adjacent tissue structures.

We claim:

1. An apparatus for thermally treating sweat glands in a volume of skin of a patient, the apparatus comprising
a unipolar RF handpiece comprising a unipolar RF electrode; and
a bipolar RF handpiece comprising a supply electrode and a ground electrode, separated by an insulator;
wherein the unipolar RF handpiece is operable to introduce RF waves from an RF energy source through the unipolar electrode into the volume of skin to dielectrically heat the sweat glands, and wherein
the bipolar RF handpiece is operable to introduce RF current from the RF energy source through the supply electrode into the biological tissue and on to the ground electrode to dielectrically heat the sweat glands.

2. The apparatus of claim 1 wherein the supply electrode is arranged coaxially inside the ground electrode, forming a uniform, homogeneous distribution of the RF current introduced by the bipolar handpiece.

3. The apparatus of claim 2 wherein the distance between the supply electrode and the ground electrode is adjustable.

4. A method for thermally treating sweat glands in a volume of skin of a patient, the method comprising:
applying RF waves to a first portion of the volume of skin to dielectrically heat the sweat glands, thereby increasing their water content; and
applying RF current to a second portion of the volume of skin, thereby further dielectrically heating those heated sweat glands common to both the first and second portions of the volume.

5. The method of claim 4 wherein the sweat glands heated by the RF current are disrupted or destroyed.

6. The method of claim 5 wherein the disruption or destruction of the sweat glands by the RF current occurs before the surrounding and or intervening tissue reaches a disruptive temperature, with the result that the treatment is effectively selective for the sweat glands.

7. A method for thermally treating sweat glands in a volume of skin of a patient, the method comprising:
applying RF waves to at least a lower part of the volume of skin to dielectrically heat the sweat glands, thereby increasing their water content; and
applying RF current to an upper part of the volume of skin, thereby slowing conductive or convective cooling of the heated lower part of the volume of skin through the upper part, so as to maintain the therapeutic heat longer in the volume without directly heating the entire volume.
